(19) United States
(12) Patent Application Publication
(10) Pub. No.: US 2023/0100536 A1
(43) Pub. Date: Mar. 30, 2023

(54) INTERCELLULAR AND INTRACELLULAR PROXIMITY-BASED LABELING COMPOSITIONS AND SYSTEMS

(71) Applicant: The Trustees of Princeton University, Princeton, NJ (US)

(72) Inventors: Aaron Trowbridge, Princeton, NJ (US); Ciaran Seath, Princeton, NJ (US); David W.C. MacMillan, Princeton, NJ (US)

(21) Appl. No.: 17/802,823

(22) PCT Filed: Feb. 26, 2021
(86) PCT No.: PCT/US2021/019959

§ 371 Date: Aug. 26, 2022

Related U.S. Application Data
(60) Provisional application No. 62/982,366, filed on Feb. 27, 2020.
(60) Provisional application No. 63/076,658, filed on Sep. 10, 2020.

(51) Int. Cl. C07D 213/22; C07D 213/56; C07D 213/79; C07D 401/14; C07F 15/00; C09K 11/06

(52) U.S. Cl. CPC C07D 213/22; C07D 213/56; C07D 213/79; C07D 401/14; C07F 15/0033; C09K 11/06; C09K 2211/1007; C09K 2211/1029; C09K 2211/185

(57) ABSTRACT

In one aspect, transition metal complexes are described herein having composition and electronic structure for generating reactive labeling intermediates having lifetimes and diffusion radii advantageous for proximity-based labeling of various biomolecular species, including proteins, in intracellular and intercellular environments.

### RELATED APPLICATION DATA

The present application claims priority pursuant to Article 8 of the Patent Cooperation Treaty to U.S. Provisional Patent Application Ser. No. 62/982,366 filed Feb. 27, 2020 and U.S. Provisional Patent Application Ser. No. 63/076,658 filed Sep. 10, 2020, each of which is incorporated herein by reference in its entirety.

### STATEMENT OF GOVERNMENT RIGHTS

This invention was made with government support under Grant No. 5R01GM103558-08 awarded by the National Institutes of Health and National Institute of General Medical Sciences. The government has certain rights in the invention.

### FIELD

The present invention relates to compositions, systems, and methods for proximity-based labeling and, in particular, to transition metal catalysts for intercellular and intracellular proximity-based labeling.

### BACKGROUND

Protein proximity labeling has emerged as a powerful approach for profiling protein interaction networks. The ability to label associated or bystander proteins through proximity labeling can have important implications on further understanding the cellular environment and biological role of a protein of interest. Current proximity labeling methods all involve the use of enzyme-generated reactive intermediates that label neighboring proteins on a few select amino acid residues through diffusion or physical contact. Despite the transformative impact of this technology, the inherent stability of these reactive intermediates such as phenoxy radicals (t_{1/2}>100 μs) through peroxidase activation or biotin-AMP (t_{1/2}>60 s) through biotin ligases can promote diffusion far from their point of origin. As a result, these enzyme-generated reactive intermediates pose a challenge to profiling within tight micro-environments. Furthermore, the large enzyme size, the dependency on certain amino acids for labeling, and the inability to temporally control these labeling systems present additional challenges for profiling within confined spatial regions. Given these limitations, new approaches for proximity-based labeling are needed.

### SUMMARY

In one aspect, transition metal complexes are described herein having composition and electronic structure for generating reactive labeling intermediates having lifetimes and diffusion radii advantageous for proximity-based labeling of various biomolecular species, including proteins. A transition metal catalyst, in some embodiments, is of Formula I.

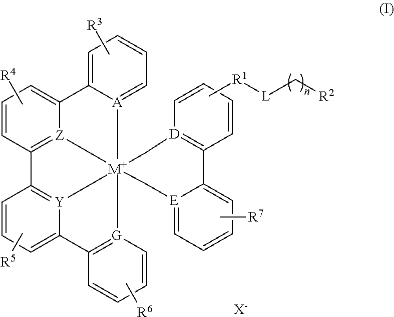

wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is a linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

As described further herein, polarity of the transition metal complexes can be tailored to specific cellular environments via selection of R³-R⁷. In some embodiments, for example, one or more of R³-R⁷ are selected to exhibit hydrophilic character via charged and/or polar chemical moieties. In such embodiments, the transition metal complex can exhibit hydrophilic character suitable for placement in intercellular or extracellular aqueous environments. Alternatively, the one or more of R³-R⁷ are selected to exhibit hydrophobic, lipophilic, or non-polar character. In some embodiments, for example, one or more of R³-R⁷ can be alkyl, fluoro, or fluoroalkyl. Transition metal complexes described herein exhibiting hydrophobic, lipophilic, or non-polar character can be suitable for placement or passage into intracellular environments. The transition metal complexes can pass through the cellular membrane for mapping local intracellular environments according to the principles described herein. Accordingly, such transition metal complexes are cell permeable.

Moreover, in some embodiments, the transition metal complex has a triplet energy state greater than 60 kcal/mol. The metal center can be selected from transition metals of the platinum group, in some embodiments. The metal center, for example, can be iridium. In some embodiments, n of Formula I is from 1 to 20.

In another aspect, compositions and methods are described herein for providing a microenvironment mapping platform operable to selectively identify various features, including protein-protein interactions on cellular membranes as well as protein, nucleic acid and/or other biomolecular interactions within cells. In some embodiments, a composition comprises a transition metal catalyst of Formula I, and a protein labeling agent, wherein the transition metal catalyst activates the protein labeling agent to a reactive intermediate. The transition metal catalyst of Formula I, in some embodiments, can have electronic structure for permitting energy transfer to the protein labeling agent to form the reactive intermediate. The reactive intermediate reacts or crosslinks with a protein or other biomolecule within the diffusion radius of the reactive intermediate. If a protein or other biomolecule is not within the diffusion radius, the reactive intermediate is quenched by the surrounding environment. As described further herein, the diffusion radius of the reactive intermediate can be tailored to specific microenvironment mapping considerations, and can be limited to the nanometer scale. In some embodiments, for example, the diffusion radius can be less than 10 nm or less than 5 nm. Moreover, in some embodiments, the reactive intermediate can have a half-life of less than 5 ns. In some embodiments, a protein labeling agent can be functionalized with a marker, such as biotin or luminescent markers for aiding in analysis. Energy transfer from the catalyst to the protein labeling agent can occur via a variety of mechanisms described further herein, including Dexter energy transfer.

In another aspect, conjugates are described herein for use in proximity-based labeling systems. A conjugate comprises a transition metal complex coupled to a biomolecular binding agent, wherein prior to coupling to the biomolecular binding agent, the transition metal complex is of Formula I described above. As detailed further herein, the biomolecular binding agent can be employed to locate the transition metal complex in the desired intracellular or intercellular/extracellular environment for proximity labeling and associated analysis. The biomolecular binding agent can exhibit selective binding to guide the conjugate to the desired location for proximity-based labeling and associated micromapping of intercellular/extracellular environments, including cellular membranes. Alternatively, the biomolecular binding agent can exhibit selective binding to guide the conjugate to the desired location for proximity-based labeling and associated micromapping of intracellular environments, including various organelle environments as well as environments local to the nucleus. The biomolecular binding agent, for example, can comprise a peptide, protein, sugar, small molecule, nucleic acid, or combinations thereof. As described further herein, the transition metal complex can comprise a reactive functionality for coupling a biomolecular binding agent, including click chemistries. In some embodiments, the transition metal complex can couple to the biomolecular binding agent in the absence of copper. Conjugates described herein can be employed with a protein labeling agent for systems for cellular proximity-based labeling detailed above.

In a further aspect, methods of proximity-based labeling are described herein. A method of proximity-based labeling comprises providing a transition metal catalyst of Formula (I), and activating a protein labeling agent to a reactive intermediate with the catalyst. The reactive intermediate couples or bonds to a protein. In some embodiments, the transition metal catalyst is coupled to a biomolecular binding agent to selectively locate or target the catalyst to a specific environment for protein mapping in conjunction with the protein labeling agent. The transition metal catalyst, conjugate, and protein labeling agent can have composition and/or properties described above and in the following detailed description.

These and other embodiments are further described in the following detailed description.

### BRIEF DESCRIPTION OF THE DRAWINGS

FIG. 1 illustrates transition metal catalysts described herein according to some embodiments.

FIG. 2 illustrates a transition metal catalyst and conjugate described herein according to some embodiments.

FIG. 3 illustrates a cell permeable conjugate comprising transition metal catalyst and JQ1 biomolecular binding agent according to some embodiments.

FIG. 4 illustrates a synthetic scheme for producing the cell permeable conjugate of FIG. 3 according to some embodiments.

FIG. 5A is a Western Blot of intercellular labeling with conjugates described herein according to some embodiments.

FIG. 5B illustrates results of densitometry analysis of the Western Blot of FIG. 5A.

FIG. 6 provides the results of time dependent labeling of BRD4 in HeLa cells.

FIG. 7 illustrates a non-cell permeable conjugate.

FIG. 8 illustrate BRD4 labeling results between the cell permeable conjugate of FIG. 3 and the non-cell permeable conjugate of FIG. 7.

FIG. 9 illustrates structure of a (−)-JQ1 conjugate and BRD4 labeling relative to a (+)-JQ1 conjugate according to some embodiments.

FIGS. 10A-10C illustrate volcano plots of significance vs. fold enrichment for targeted bromodomain proteins with a conjugate described herein according to some embodiments.

FIG. 11 illustrates a synthetic pathway for a conjugate described herein according to some embodiments.

FIG. 12 provides a volcano plot of significance vs. fold enrichment for targeted tubulin proteins in MCF-7 cells using the cell permeable conjugate of FIG. 11 according to some embodiments.

FIG. 13 illustrates confocal microscopy images of intracellular labeling by the conjugate of FIG. 3 at differing time points, according to some embodiments.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents.

The term “alkynyl” as used herein, alone or in combination, refers to a straight or branched chain hydrocarbon group having at least one carbon-carbon triple bond and optionally substituted with one or more substituents.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, boron, oxygen and/or sulfur.

The term “heterocycle” as used herein, alone or in combination, refers to an mono- or multicyclic ring system in which one or more atoms of the ring system is an element other than carbon, such as boron, nitrogen, oxygen, and/or sulfur or phosphorus and wherein the ring system is optionally substituted with one or more ring substituents. The heterocyclic ring system may include aromatic and/or non-aromatic rings, including rings with one or more points of unsaturation.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as boron, nitrogen, oxygen, sulfur or phosphorus, alone or in combination, and wherein the ring system is optionally substituted with one or more ring substituents.

The term “alkoxy” as used herein, alone or in combination, refers to the moiety RO—, where R is alkyl, alkenyl, or aryl defined above.

The term “halo” as used herein, alone or in combination, refers to elements of Group VIIA of the Periodic Table (halogens). Depending on chemical environment, halo can be in a neutral or anionic state.

Terms not specifically defined herein are given their normal meaning in the art.

### I. Transition Metal Complexes

In one aspect, transition metal complexes are described herein having composition and electronic structure for generating reactive labeling intermediates having lifetimes and diffusion radii advantageous for proximity-based labeling of various biomolecular species, including proteins. A transition metal catalyst, in some embodiments, is of Formula I.

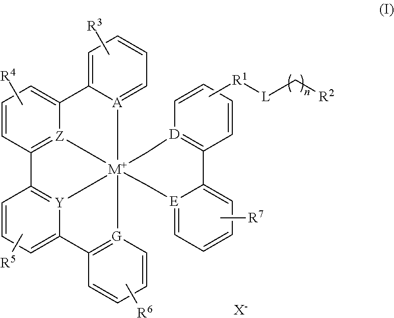

wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is a linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

It is understood that hydrogen occupies positions on the aryl rings of Formula I in the absence of optional substituents R³-R⁷. Additionally, in some embodiments, counterion (X⁻) can be selected from tetraalkylborate, tetrafluoroborate, tetraphenylborate, PF₆⁻, and chloride.

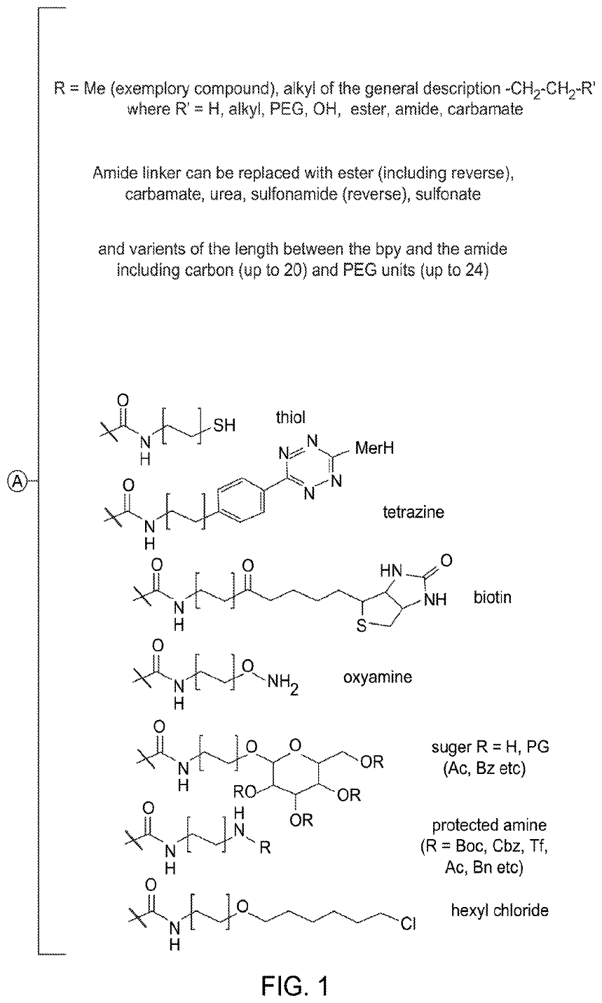

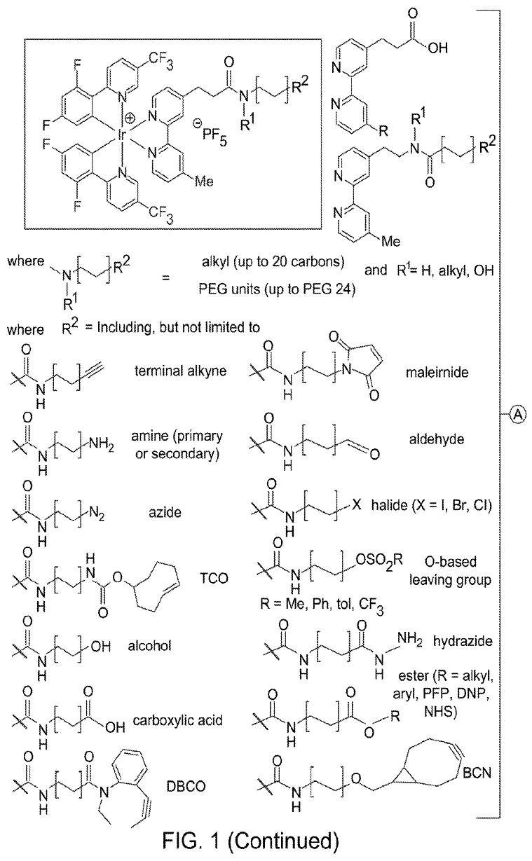

Polarity of the transition metal complexes can be tailored to specific cellular environments via selection of R³-R⁷. In some embodiments, for example, one or more of R³-R⁷ are selected to exhibit hydrophilic character via charged and/or polar chemical moieties. In such embodiments, the transition metal complex can exhibit hydrophilic character suitable for placement in intercellular/extracellular environments. Transition metal complexes illustrated in FIG. 2, for example, incorporate charged and polar chemical moieties for the aqueous intercellular environment. Alternatively, the one or more of R³-R⁷ are selected to exhibit hydrophobic, lipophilic, or non-polar character. In some embodiments, for example, one or more of R³-R⁷ can be alkyl, fluoro, or fluoroalkyl. FIG. 1 illustrates one non-limiting embodiment of a transition metal complex comprising alkyl, fluoro, or fluoroalkyl substituents. Transition metal complexes described herein exhibiting hydrophobic, lipophilic, or non-polar character can be suitable for placement in intracellular environments. As demonstrated in the examples herein, the transition metal complexes can pass through the cellular membrane for mapping local intracellular environments according to the principles described herein. In some embodiments, for example, a cell permeable transition metal complex of Formula I has an aqueous solubility less than 150 μM at 0.2% DMSO in pure water. In some embodiments, a transition metal complex of Formula I has an aqueous solubility of less than 100 μM. A transition metal complex of Formula I exhibiting hydrophobic, lipophilic, or non-polar character can have aqueous solubility of 1 μM to 150 μM or 1 μM to 100 μM at 0.2% DMSO in pure water. Aqueous solubility can be determined according to retention times of the transition metal complexes on a C18 column (HPLC). The foregoing aqueous solubility values can also apply to conjugates described herein comprising the transition metal complex coupled to a biomolecular binding agent.

Transition metal catalysts described herein are employed in compositions for providing microenvironment mapping platforms operable to selectively identify various features, including protein-protein interactions on cellular membranes. In some embodiments, a composition comprises a transition metal catalyst of Formula I, and a protein labeling agent, wherein the transition metal catalyst activates the protein labeling agent to a reactive intermediate. The transition metal catalyst of Formula I, in some embodiments, can have electronic structure for permitting energy transfer to the protein labeling agent to form the reactive intermediate. The reactive intermediate reacts or crosslinks with a protein or other biomolecule within the diffusion radius of the reactive intermediate. If a protein or other biomolecule is not within the diffusion radius, the reactive intermediate is quenched by the surrounding environment.

The energy transfer to the protein labeling agent can originate from an excited state of the transition metal catalyst electronic structure, in some embodiments. The excited state of the catalyst, for example, can be a singlet excited state or triplet excited state. The excited state of the catalyst can be generated by one or more mechanisms, including energy absorption by the catalyst. In some embodiments, the catalyst is a photocatalyst, wherein the excited state is induced by absorption of one or more photons. In other embodiments, the catalyst may be placed in an excited state by interaction with one or more chemical species in the surrounding environment. Alternatively, the energy transfer to the protein labeling agent, including electron transfer, may originate from a ground state of the catalyst electronic structure.

Energy transfer, including electron transfer, to the protein labeling agent forms a reactive intermediate of the protein labeling agent. The reactive intermediate reacts or crosslinks with a protein or other biomolecule within the diffusion radius of the reactive intermediate. If a protein or other biomolecule is not within the diffusion radius, the reactive intermediate is quenched by the surrounding environment. The diffusion radius of the reactive intermediate can be tailored to specific microenvironment mapping (proximity-based labeling) considerations, and can be limited to the nanometer scale. In some embodiments, for example, the diffusion radius of the reactive intermediate can be less than 10 nm, less than 5 nm, less than 4 nm, less than 3 nm, or less than 2 nm prior to quenching in the surrounding environment. Accordingly, the reactive intermediate will react or crosslink with a protein or other biomolecule within the diffusion radius or be quenched by the surrounding environment if no protein or biomolecule is present. In this way, high resolution of the local environment can be mapped via concerted effort between the catalyst and protein labeling agent. Additionally, the reactive intermediate can exhibit a t_{1/2} less than 5 ns, less than 4 ns, or less than 2 ns prior to quenching, in some embodiments. In additional embodiments, the diffusion radius can be extended to between 5-500 nm though extension of the reactive intermediate half-life.

Any transition metal catalyst-protein labeling agent combination exhibiting the foregoing electronic structure properties for energy transfer and reactive intermediate generation and associated protein or biomolecule binding can be employed for microenvironment mapping. A transition metal complex of Formula I, in some embodiments, can exhibit a long-lived triplet excited state (T₁) facilitating energy transfer to the protein labeling agent. The T₁ state can have t_{1/2} of 0.2-2 μs, for example. Transition metal complexes described herein can be photocatalytic and, in some embodiments, absorb light in the visible region of the electromagnetic spectrum. Absorption of electromagnetic radiation can excite the transition metal complex to the S₁ state followed by quantitative intersystem crossing to the T₁ state. The transition metal catalyst can subsequently undergo short-range Dexter energy transfer to a protein labeling agent, and returned to the ground state, S₀. The energy transfer to the labeling agent activates the labeling agent for reaction with a protein or other biomolecule. The T₁ state of the transition metal complex can be greater than 60 kcal/mol, in some embodiments. The metal center, for example, can be selected from transition metals of the platinum group. The metal center can be iridium, in some embodiments.

FIGS. 1 and 2 illustrate various transition metal complexes described herein. As illustrated in FIG. 1, R² can be selected as a reactive functionality for coupling a biomolecular binding agent. In some embodiments, for example, R² comprises one or more click chemistry moieties including, but not limited to, BCN, DBCO, TCO, tetrazine, alkyne, and azide. As illustrated in FIG. 1, these click chemistries of R² can be directly coupled to the linker (L) or coupled via a heteroatom, aryl, or carbonyl.

Protein labeling agents receive energy transfer from the transition metal catalyst to form a reactive intermediate. The reactive intermediate reacts or crosslinks with a protein or other biomolecule within the diffusion radius of the reactive intermediate. Diffusion radii of reactive intermediates are described above. Specific identity of a protein labeling agent can be selected according to several considerations, including identity of the catalyst, the nature of the reactive intermediate formed, lifetime and diffusion radius of the reactive intermediate.

For example, in embodiments wherein the transition metal catalyst is a photocatalyst, the protein labeling agent can be a diazirine. Triplet energy transfer from the excited state photocatalyst can promote the diazirine to its triplet (T₁) state. The diazirine triplet under-goes elimination of N₂ to release a free triplet carbene, which undergoes picosecond-timescale spin equilibration to its reactive singlet state (t_{1/2}<1 ns) which either crosslinks with a nearby protein or is quenched in the aqueous environment. In some embodiments, the extinction coefficient of the transition metal complex is 3 to 5 orders of magnitude greater than that of the diazirine.

Any diazirine consistent with the technical principles discussed herein. Diazirine sensitization, for example, can be extended to a variety of p- and m-substituted aryltrifluoromethyl diazirines bearing valuable payloads for microscopy and proteomics applications, including free carboxylic acid, phenol, amine, alkyne, carbohydrate, and biotin groups. The diazirine can be functionalized with a marker, such as biotin. In some embodiments, the marker is desthiobiotin. The marker can assist in identification of proteins labeled by the protein labeling agent. The marker, for example, can be useful in assay results via western blot and/or other analytical techniques. Markers can include alkyne, azide, FLAG tag, fluorophore, and chloroalkane functionalities, in addition to biotin and desthiobiotin.

In some embodiments wherein the transition metal catalyst is a photocatalyst, the protein labeling agent can be an azide. Triplet energy transfer from the excited state photocatalyst can promote nitrene formation from the azide. The reactive nitrene either crosslinks with a nearby protein or is quenched in the aqueous environment. Any azide operable to undergo energy transfer with eth transition metal photocatalyst for nitrene formation can be employed. In some embodiments, an azide is an aryl azide.

### II. Conjugates

In another aspect, conjugates are described herein for use in proximity-based labeling systems. A conjugate comprises a transition metal complex coupled to a biomolecular binding agent, wherein prior to coupling to the biomolecular binding agent, the transition metal complex is of Formula I described above. As detailed further herein, the biomolecular binding agent can be employed to locate the transition metal catalyst in the desired cellular environment for proximity labeling and associated analysis and mapping. In some embodiments, the desired cellular environment is intercellular. In other embodiments, the desired environment is intracellular. The biomolecular binding agent can exhibit selective binding to guide the conjugate to the desired location for proximity-based labeling and associated micromapping of intercellular environments.

The transition metal complex of the conjugate can comprise any transition metal complex having structure and/or properties described in Section I above. Moreover, the biomolecular binding agent can comprise a multivalent display system comprising a protein, polysaccharide, or nucleic acid. In some embodiments, the biomolecular binding agent is biotin or a small molecule ligand with a specific binding affinity for a target protein. The biomolecular binding agent, for example, can be an antibody. In some embodiments, the biomolecular binding agent is a secondary antibody for interacting with a primary antibody bound to the desired antigen. Additionally, the biomolecular binding agent may be covalently coupled to the photocatalytic transition metal complex.

The biomolecular binding agent can be bonded to the transition metal catalyst. In some embodiments, the catalyst comprises a reactive handle or functionality for coupling the biomolecular binding agent. In some embodiments, for example, a catalyst can comprise one or more click chemistry moieties including, but not limited to, BCN, DBCO, TCO, tetrazine, alkyne, and azide. FIGS. 1 and 2 illustrate various transition metal photocatalysts of Formula (I) having a reactive functionality for coupling a biomolecular binding agent. As illustrated in FIGS. 1 and 2, a linker of varying length can be employed between the reactive functionality and the coordinating ligand. Length of the linker, such as an amide or polyamide linker, can be chosen according to several considerations, including steric condition of the target site. Moreover, in some embodiments, the transition metal complex can couple to the biomolecular binding agent in the absence of copper.

In some embodiments, conjugates exhibit polarity suitable for labeling applications in intercellular environments. Alternatively, the conjugates can be cell permeable, wherein the conjugates can pass through the cell membrane for intracellular labeling applications. In some embodiments, for example, conjugates can exhibit the aqueous solubility values recited in Section I above for the cell permeable transition metal complexes.

### III. Systems for Intracellular Proximity-Based Labeling

In another aspect, systems for proximity-based labeling are described herein. A system, for example, comprises a protein labeling agent, and a transition metal catalyst, wherein the transition metal catalyst has electronic structure permitting electron transfer to the protein labeling agent to provide a reactive intermediate. The reactive intermediate can subsequently couple to a protein or other biomolecule in the local or immediate cellular environment. In some embodiments, the transition metal complex is for Formula I described herein.

In some embodiments, the electron transfer originates from an excited state of the catalyst electronic structure, including a singlet excited state or triplet excited state. The excited state of the catalyst, for example, can be photo-induced, in some embodiments. Alternatively, the electron transfer may originate from a ground state of the catalyst electronic structure.

As described herein, electron transfer to the protein labeling agent provides a reactive intermediate. The reactive intermediate can exhibit a diffusion radius consistent with the proximity labeling embodiments detailed herein. Diffusion radius can be limited or bounded by rapid quenching of the reactive intermediate by the surrounding aqueous environment. For example, the reactive intermediate may have a diffusion radius less than 5 nm, less than 3 nm or less than 2 nm prior to quenching in an aqueous environment. Accordingly, the reactive intermediate will react or crosslink with a protein or other biomolecule within the diffusion radius or be quenched by the aqueous environment if no protein or biomolecule is present. In this way, high resolution of the local environment can be mapped via concerted effort between the catalyst and protein labeling agent. Additionally, the reactive intermediate can exhibit a t_{1/2} less than 2 ns prior to quenching, in some embodiments. In additional embodiments, the diffusion radius can be extended to between 5-500 nm though extension of the reactive intermediate half-life.

Any catalyst-protein labeling agent combination exhibiting the foregoing electronic structure properties for electron transfer and reactive intermediate generation can be employed for microenvironment mapping. In some embodiments, the catalyst-protein labeling agent combination comprises transition metal catalyst of Formula I and diazirine labeling agent. A transition metal catalyst of Formula I can have any structure and/or properties described in Section I above. In some embodiments, a protein labeling agent can be functionalized with a marker, such as biotin or luminescent markers for aiding in analysis. Diazirine sensitization could be extended to a variety of p- and m-substituted aryltrifluoromethyldiazirines bearing valuable payloads for microscopy and proteomics applications, including free carboxylic acid, phenol, amine, alkyne, carbohydrate, and biotin groups. The extinction coefficient of the transition metal catalyst can be five orders of magnitude larger than that of the diazirine at the wavelength emit-ted by blue LEDs used for sensitization (450 nm), explaining the absence of a background non-catalyzed reaction.

In some embodiments, multiple protein labeling agents can be employed with the transition metal catalyst. In such embodiments, the transition metal catalyst exhibits electronic structure to permit electron transfer to one or all of the protein labeling agents to provide reactive intermediates. The reactive intermediates can exhibit different diffusion radii, in some embodiments, thereby binding to different proteins or biomolecules at different locations. Such embodiments can enhance resolution of intracellular proximity-based labeling systems described herein.

Additionally, the transition metal complex in systems contemplated herein can be coupled to a biomolecular binding agent to provide a conjugate, as described in Section II above. Inclusion of the biomolecular binding agent can direct the transition metal catalyst to the desired cellular environment for analysis and mapping in association with the one or more protein binding agents. In some embodiments, a system described herein can employ multiple conjugates and protein labeling agents, wherein each conjugate and associated protein labeling agent are specific to different intracellular environment.

### IV. Methods of Intracellular Proximity-Based Labeling

In a further aspect, methods of cellular proximity-based labeling are described herein. In some embodiments, a method comprises providing a protein labeling agent and a conjugate comprising a transition metal catalyst coupled to a biomolecular binding agent. The protein labeling agent is activated to a reactive intermediate by the transition metal catalyst, and the reactive intermediate couples to a protein or other biomolecule in the cellular environment. Methods described herein can further comprise detecting or analyzing the protein couples to the reactive intermediate, resulting in mapping of a local cellular environment.

The protein labeling agent and conjugate can have any structure, composition, and/or properties described in any of Sections I-III above.

These and other embodiments are further illustrated in the following Examples.

### Example 1—Transition Metal Catalyst

### Step 1

### 3-(4′-Methyl-[2,2′-bipyridin]-4-yl)propanoic acid

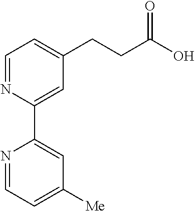 [SMILES: Cc1ccnc(-c2cc(CCC(=O)O)ccn2)c1]

3-(4′-Methyl-[2,2′]bipyridinyl-4-yl)-propionic acid ethyl ester 4,4′-Dimethyl-2,2′-bipyridyl (2.5 g, 13.5 mmol) was dissolved in dry THE (20 mL) under a nitrogen atmosphere, in a flame-dried flask. The solution was cooled to −78° C., and a solution of LDA (14.8 mmol, 1.1 equiv) was added. The reaction mixture was allowed to warm to room temperature for 1.5 hours. This solution was cannulated into a solution of ethyl 2-bromoacetate (2.3 ml, 20 mmol) in dry THE (15 ml) at −78° C. under N2. The reaction mixture was allowed to reach room temperature slowly overnight and quenched by addition of sat. sodium bicarbonate solution. Work-up using ethyl acetate followed by drying over Na₂SO₄ and concentration under reduced pressure provided the crude product. The crude residue was purified by column chromatography (Silica gel; DCM:MeOH:NH₄OH 95:5:0.5) to provide the desired product in 69% yield.

### Step 2: 5-(4′-Methyl-[2,2]bipyridinyl-4-yl)-pent-4-enoic acid

The bipyridinyl ethyl ester from step 1 was taken up in 1:1 THF:water before the addition of LiOH (2 equiv.). The reaction mixture was stirred at room temperature for 16 h (completion by TLC) before being quenched through the addition of NH₄Cl (until pH 5-6). The mixture the extracted with EtOAc, dried over Na₂SO₄ and concentrated under reduced pressure to provide the desired product as an off-white powder (63% yield).

### tert-Butyl (2-(3-(4′-methyl-[2,2′-bipyridin]-4-yl)propanamido)ethyl)carbamate

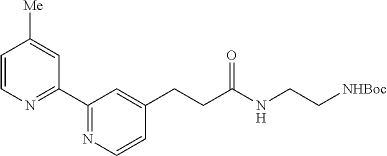 [SMILES: Cc1ccnc(-c2cc(CCC(=O)NCCNC(=O)OC(C)(C)C)ccn2)c1]

To a 20 mL vial charged with bipy x (228 mg, 1 mmol, 1 equiv), PyBOP (612 mg, 1.2 mmol, 1.2 equiv.), and tert-butyl (2-aminoethyl)carbamate (192 mg, 1.2 mmol, 1.2 equiv.) was added DMF (2 mL) then diisopropylethylamine (347 μL, 0.15 mmol, 3 equiv.). The reaction was stirred for 16 hours. The resulting mixture was quenched through the addition of water and EtOAc. The layers were separated, and the organics were washed with saturated NaHCO₃ and, H₂O, and brine. The organic layer was then dried over Na₂SO₄ and concentrated under reduced pressure to provide the a yellow oil that was purified by flash column chromatography (silica gel, 0-15% MeOH/CH₂Cl₂) to provide the desired compound as a yellow solid (380 mg, 99%).

### Ir-Catalyst X

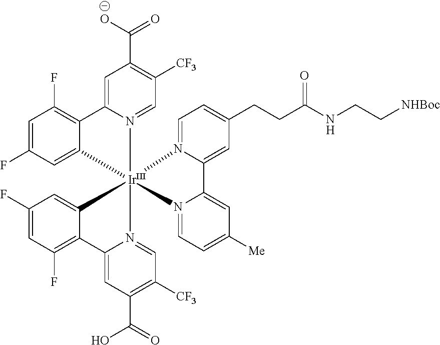

A round bottomed flask charged with bipy y (161 mg, 0.42 mmol, 1.05 equiv.) and Ir[dF(CO₂H—CF₃)ppy]MeCN₂ (351 mg, 0.4 mmol, 1 equiv.) was added DCM/EtOH (4 mL, 4:1) and the reaction mixture was stirred at 30° C. for 16 hours. The resulting solution was concentrated under reduced pressure directly onto silica gel. The crude product was purified by flash column chromatography (silica gel, 0-25% MeOH/DCM) to provide the desired Ir-catalyst (200 mg, 42% yield).

### DBCO Ir Catalyst

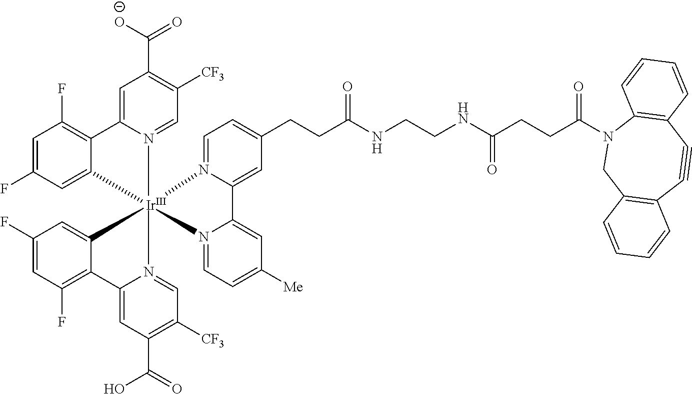

To a 5 mL vial (wrapped in black tape to obscure light) charged with Ir-cat X (9.4 mg, 0.008 mmol, 1 equiv) in CH₂Cl₂ (500 μL) mg, was cooled to 0° C. before the addition of trifluoroacetic acid (100 μL). The reaction mixture was warmed to room temperature and stirred until completion (monitored by TLC and HRMS). The completed reaction was concentrated under reduced pressure and the solid was slurried with MeOH and concentrated under reduced pressure (performed 3 times or more to remove excess acid).

The Ir catalyst-trifluoroacetic acid salt was then dissolved in DMF (500 μL) before the addition of diisopropylethylamine (10 μL). To this solution was added DBCO-NHS (6 mg, 0.016 mmol, 2 equiv.) and the solution was stirred in the dark for 3 h. Upon completion, (by HRMS/TLC) the reaction mixture was directly purified by flash column chromatography (C18, 5-95% MeCN/H₂O) to provide the desired compound as a yellow solid (10 mg, 91%).

### Example 2—Transition Metal Catalyst

Step 1: A round bottomed flask charged with 3-(4′-methyl-[2,2′-bipyridin]-4-yl)propanoic acid and Ir[dF(CF₃)ppy]MeCN₂ PF₆ was added MeCN/H₂O (4:1) and the reaction mixture was stirred at 70° C. for 16 hours. The resulting solution was concentrated under reduced pressure to provide a yellow solid. The crude product was purified by flash column chromatography (silica gel, 0-10% MeOH/DCM) to provide the desired acid bearing Ir-catalyst (55% yield).

Step 2 (for differentially activated catalysts): To a 20 mL vial charged with Ir-catalyst, PyBOP, and amine was added DMF. The reaction mixture was sparged with N₂ for 10 minutes in the dark before the addition of diisopropylethylamine. The reaction was stirred in the dark under an atmosphere of N2 for 16 hours. The resulting mixture was quenched through the addition of water and EtOAc. The layers were separated, and the organics were washed with 5% citric acid, saturated NaHCO₃ and brine. The organic layer was then dried over Na₂SO₄ and concentrated under reduced pressure to provide the desired compound.

### Example 3—Cell Permeable Conjugate, (+)-JQ1-PEG3-Ir

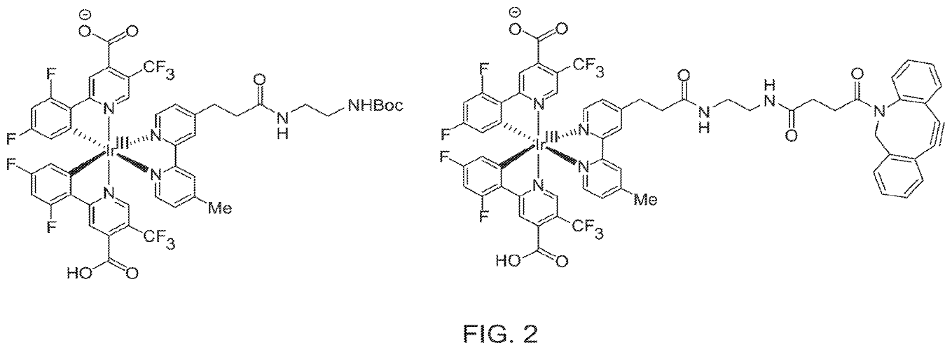

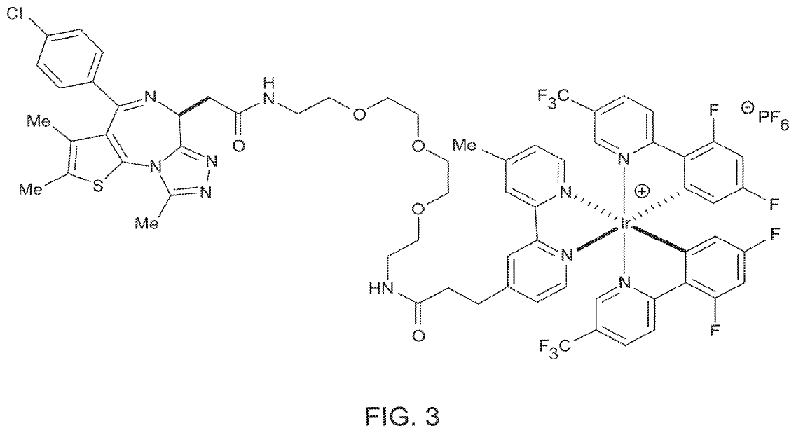

The cell permeable conjugate comprising the transition metal complex and JQ1 biomolecular binding agent of FIG. 3 was synthesized according to the following protocol. The synthetic scheme for the transition metal complex and JQ1 biomolecular binding agent is also illustrated in FIG. 4. To a stirred solution of (+)-JQ1-CO₂H (177 mg, 0.44 mmol) in anhydrous DMF (4.5 mL) was added HATU (176 mg, 0.46 mmol) followed by DIPEA (230 μL, 1.32 mmol). The reaction was stirred at room temperature for 10 minutes under N2 and a solution of t-Boc-N-amido-PEG3-amine (143 mg, 0.49 mmol) in anhydrous DMF (0.5 mL) was added dropwise. The resulting mixture was stirred overnight, diluted with EtOAc, and quenched by the addition of saturated aqueous NaHCO₃. The aqueous phase was removed and the organic layer washed with additional saturated aqueous NaHCO₃, brine, and dried over Na₂SO₄. The solvent was removed in vacuo, and the crude material purified by silica column chromatography (gradient elution: 0 to 10% MeOH/CH₂Cl₂) to afford (+)-JQ1-PEG3-NHBoc as a tan solid (171 mg, 57%). ¹H NMR (500 MHz, CDCl₃) δ: 7.39 (d, J=8.5 Hz, 2H), 7.31 (d, J=8.7 Hz, 2H), 7.20 (br. s, 1H), 5.35 (br. s, 1H), 4.65 (t, J=7.1 Hz, 1H), 3.69-3.46 (m, 15H), 3.36 (dd, J=15.0, 6.8 Hz, 1H), 3.30 (m, 2H), 2.65 (s, 3H), 2.39 (s, 3H), 1.66 (s, 3H), 1.41 (s, 9H). ¹³C NMR (125 MHz, CDCl₃) δ: 170.7, 164.0, 156.3, 155.7, 150.0, 136.9, 136.7, 132.2, 131.0, 131.0, 130.6, 130.0, 128.8, 79.2, 70.6, 70.6, 70.4, 70.2, 70.0, 54.5, 40.4, 39.5, 39.0, 28.5, 14.5, 13.2, 11.9. m/z HRMS found [M]⁺=675.29120, [C₃₂H₄₄ClF₁₀N₆O₆S]⁺ requires 675.27226.

To a stirred solution of (+)-JQ1-PEG3-NHBoc (146 mg, 0.22 mmol) in CH₂Cl₂ (2 mL) at 0° C. was added TFA (3 mL) dropwise. The reaction mixture was warmed to room temperature overnight and the solvent removed in vacuo. The crude mixture was basified using saturated aqueous NaHCO₃, extracted with CH₂C12, and the solvent removed in vacuo to afford (+)-JQ1-PEG3-NH₂ as a tan solid (125 mg, 99%), which was used immediately without further purification.

To a stirred solution of (+)-JQ1-PEG3-NH₂ (32 mg, 56 μmol), Ir—CO₂H (61 mg, 56 μmol), and PyBOP (45 mg, 86 μmol) in anhydrous DMF (2 mL) under N₂ in the dark was added DIPEA (30 μL, 172 μmol). The resulting mixture was stirred overnight, diluted with EtOAc, and quenched by the addition of saturated aqueous NaHCO₃. The aqueous phase was removed and the organic layer washed with additional saturated aqueous NaHCO₃, 5% aqueous citric acid, brine, and dried over Na₂SO₄. The solvent was removed in vacuo, and the crude material purified by silica column chromatography (gradient elution: 0 to 3% MeOH/CH₂Cl₂) and C8 reverse phase preparative HPLC (gradient elution: 30 to 100% MeCN/H₂O (0.1% formic acid)) to afford (+)-JQ1-PEG3-iridium as a yellow solid (25 mg, 27%). ¹H NMR (500 MHz, CDCl₃) δ: 9.24-8.92 (m, 2H), 8.58-8.27 (m, 2H), 8.24 (s, 1H), 8.04 (dd, J=12.2, 8.9 Hz, 2H), 7.79-7.66 (m, 2H), 7.62 (s, 1H), 7.55 (s, 1H), 7.49 (t, J=5.1 Hz, 1H), 7.41 (d, J=8.2 Hz, 2H), 7.31 (d, J=8.2 Hz, 2H), 6.63 (dd, J=12.2 8.8 Hz, 2H), 5.62 (dd, J=8.0, 2.3 Hz, 2H), 4.80 (br. s, 2H), 4.66 (t, J=6.9 Hz, 1H), 3.70-3.30 (m, 18H), 3.24-3.15 (m, 2H), 2.93-2.77 (m, 2H), 2.66 (s, 3H), 2.63 (s, 3H), 2.39 (s, 3H), 1.66 (s, 3H). ¹³C NMR (125 MHz, CDCl₃) δ: 171.9, 170.8, 167.0 (dd, J=258.2, 16.8 Hz), 163.9, 162.7 (dd, J=262.6, 14.2 Hz), 157.6, 155.6 (d, J=9.9 Hz), 155.1 (dd, J=6.9, 28.6 Hz), 154.5, 149.6, 149.1, 145.2 (d, J=3.2 Hz), 136.8 (d, J=2.8 Hz), 136.6, 131.1, 130.9, 130.7, 130.0, 129.9, 129.6, 128.8, 127.9, 126.6, 126.4, 126.2, 123.7 (d, J=22.5 Hz), 121.7 (dd, J=273.3, 8.9 Hz), 114.2 (ddd, J=17.1, 10.1, 2.6 Hz), 100.1 (dt, J=27.0, 9.7 Hz), 70.7, 70.4, 70.3, 69.9, 69.8, 54.5, 39.6, 39.2, 38.9, 35.2, 32.1, 29.8, 29.5, 22.8, 21.8, 14.6, 14.3, 14.3, 13.2, 12.0. ¹⁹F NMR (376 MHz, CDCl₃) δ: −62.7 (d, J=3.1 Hz), −62.7 (s), −72.1 (d, J=714.3 Hz), −101.6 (dtt, J=59.3, 12.5, 8.8 Hz), −105.9-−106.1 (m). m/z HRMS found [M]⁺=1507.34161 (100), 1508.33996 (84), 1505.33212 (63), 1506.3 3296 (52), 1509.33644 (72), 1510.33378 (47) [C₆₅H₅₇ClF₁₀IrN₁₀O₅S]⁺ requires 1507.33876 (100), 1508.34202 (70), 1505.33634 (60), 1506.33969 (42), 1509.33572 (32), 1509.34538 (24), 1510.33907 (23). HPLC (Vydac 218TP C18 HPLC, gradient: 0-90% MeCN/H₂O (0.1% TFA) 10 minutes, 5 minutes 90% MeCN (0.1% TFA), 1 mL/min, 254 nm): τᵣ=12.5 min.

The enantiomer was prepared analogously from (−)-JQ1-CO₂H.

### Example 4—Intracellular Microenvironmental Mapping

### In-Cell Labelling:

To HeLa cells in 12×10 cm plates at 80% confluency in DMEM with no phenol red (Gibco) (4 mL) was added JQ1-PEG3-Ir (Example 3) (5 μM) (4 plates, A); Ir-PEG3-NHBoc (5 μM) (4 plates, B); and DMSO (4 plates, C). The plates were incubated at 37° C. for 3 hours and the media removed and replaced. Diazirine-PEG3-bioitin was added (250 μM) and the plates incubated at 37° C. for an additional 20 minutes. The plates were subsequently irradiated (without the lid) in the bioreactor at 450 nM for 15 minutes. The media was removed and the cells washed twice with cold DPBS (4° C.). The cells were resuspended in cold DPBS (4° C.), scraped and transferred to a separate 50 mL falcon tube. The cells were pelleted (1000 g for 5 minutes at 4° C.) and suspended in 1 mL of cold RIPA buffer containing PMSF (1 mM) and cOmplete EDTA free protease inhibitor (1×) (Roche). The lysed cells were incubated on ice for 5-10 minutes and sonicated (35%, 5×5 s with 30 s rest). The lysate was then centrifuged at 15×1000 g for 15 mins at 4° C. and the supernatant collected. The concentration of the cell lysate was measured by BCA assay and adjusted accordingly to equal concentration of 1 mg/mL. A control sample was removed from each plex (15 μL) and stored at −20° C. for later analysis.

### Streptavidin Pull-Down:

Magnetic Streptavidin beads (NEB) were removed (250 μL per plex) and washed twice with RIPA (0.5 mL) (5 minutes incubation on a rotisserie). The beads were pelleted on a magnetic rack, diluted with the samples (1 mL) and incubated on a rotisserie at 4° C. overnight. The beads were pelleted on a magnetic rack, the supernatant removed, and a control sample from each plex (15 μL) and stored at −20° C. for later analysis. The beads were subsequently washed with 1 x RIPA (0.5 mL), 3×1% SDS in DPBS (0.5 mL), 3×1M NaCl in DPBS (0.5 mL), 3×10% EtOH in DPBS and 1×RIPA (0.5 mL). The samples were incubated with each wash for 5 minutes prior to pelleting. The beads were resuspended in RIPA buffer (300 μL) and transferred to a new 1.5 mL Lo-bind tube.

### Western Blot Analysis:

Following the final wash and transfer procedure for pull-down, the beads were pelleted on a magnetic rack and the supernatant removed. The beads were gently centrifuged to gather at the bottom of the tube and freshly prepared elution buffer (30 mM biotin, 6 M urea, 2 M thiourea, 2% SDS in DPBS, pH=11.5) (24 μL) and 4× Laemlli buffer with BME (6 μL) was added with gentle mixing. The beads were heated to 95° C. for 15 minutes, pelleted on a magnetic rack, and the supernatant was removed while hot and beads discarded. The samples were cooled to room temperature and centrifuged. The samples (17 μL) were subsequently loaded onto a BioRad Criterion 4-20% tris-glycine gel, alongside all of the appropriate controls, and run in freshly prepared Tris running buffer (160V, 60 minutes). The gel was washed (3× MiliQ water) and transferred via iBlot 2 to an NC membrane. The membrane was again washed (3× MiliQ water) and blocked with Li-COR TBS Blocking Buffer for 1 hour at room temperature and then incubated with anti-BRD4 (A-7, Santa Cruz) (1:500) and anti-histone H3 (polyclonal Invitrogen PA5-16183) (1:2000) overnight in Pierce Protein-Free Blocking (1:2000) at 4° C. overnight. The membrane was washed 3×TBST (5 mins per wash) and 5× MiliQ water and resuspended in Pierce Protein-Free Blocking Buffer with Li-COR secondary antibodies (Goat-anti-Mouse 800) and (Goat-anti-Rabbit 700) and rocked for 1 hour at room temperature (1:12, 500). The membrane was washed 3×TBST (5 mins per wash) and 5× MiliQ water and imaged.

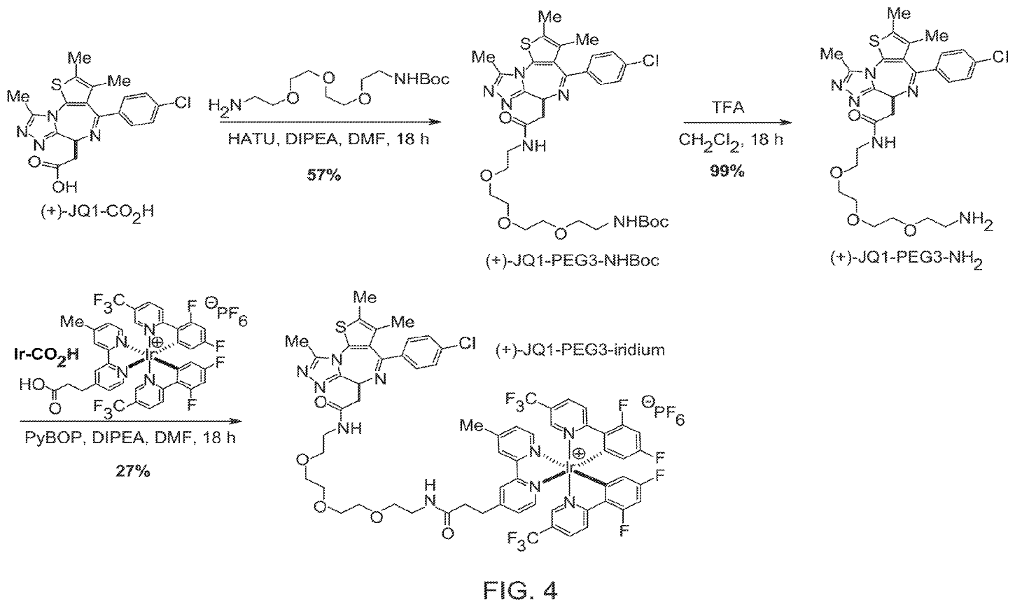

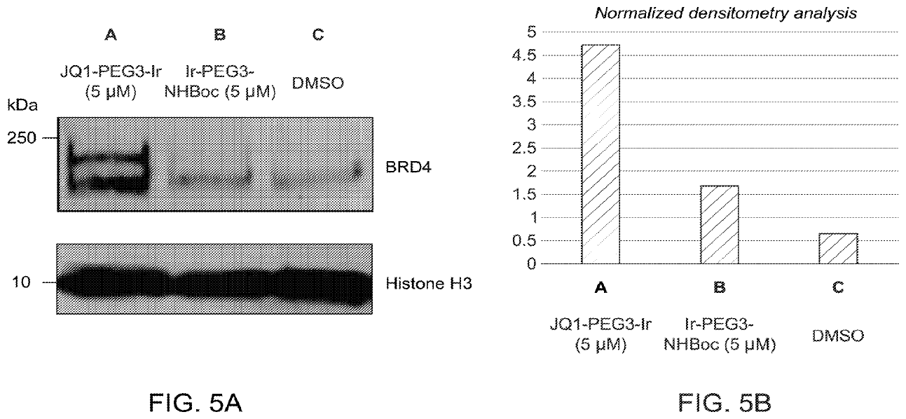

FIG. 5A illustrates the results from the Western Blot analysis, and FIG. 5B provides densitometry results of the Western Blot quantifying association of the transition metal complex with BRD4 protein. As illustrated in FIG. 5B, the cell permeable conjugate (+)-JQ1-PEG3-Ir of Example 3 herein exhibited greater than a 2.5 fold increase in labeling of BRD4 relative transition metal complex lacking the biomolecular binding agent.

### Example 5—Time Dependent Labelling of BRD4 Using (+)-JQ1-PEG3-Ir

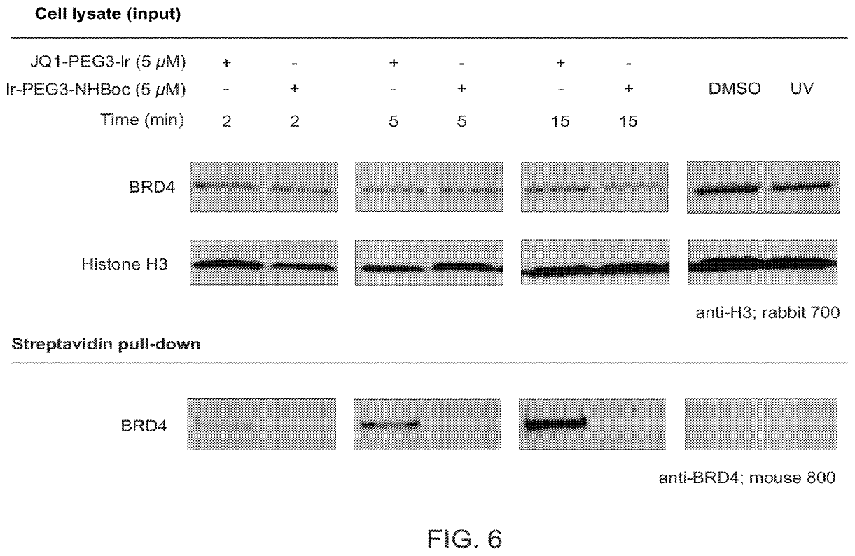

Following the in-cell labelling protocol as described in Example 4. Irradiation time was varied so as to demonstrate the degree of biotinylation over time (2, 5, and 15 minutes). The control reaction using UV light was performed using a UV-photobox wherein the plates were irradiated using 254 nm light at 4° C. for 20 minutes. FIG. 6 provides the results of time dependent labeling of BRD4 in HeLa cells. As illustrated in FIG. 6, the cell permeable conjugate synthesized in Example 3 herein enabled labeling of BRD4 at time periods of 2, 5 and 15 minutes. In contrast, transition metal catalyst not functionalized with the JQ1 biomolecular binding agent failed to produce BRD4 labeling.

### Example 6—Comparison of Labeling Between Cell Permeable and Non-Cell Permeable Conjugates

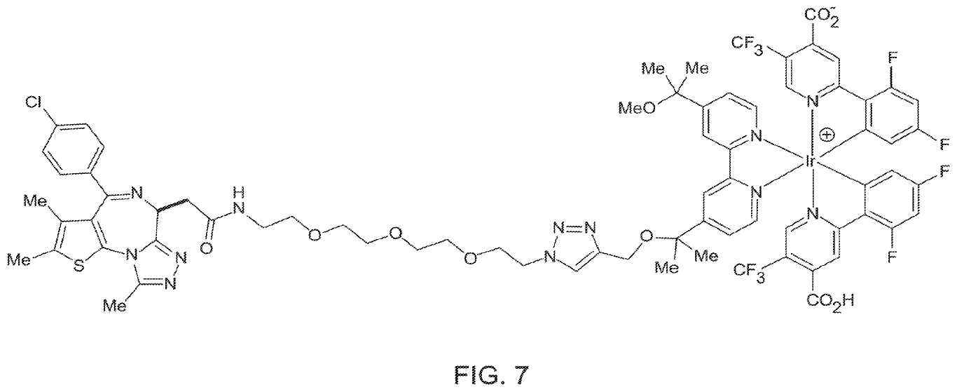

A non-cell permeable conjugate of FIG. 7 was produced as follows. The non-cell permeable conjugate is labeled JQ1-(Gen1)-Ir for the purposes of this example. (+)-JQ1-CO₂H (100 mg, 0.25 mmol), azido-PEG3-amine (60 mg, 0.27 mmol), 1-propanephosphonic anhydride (300 μL, 0.5 mmol, 50% solution in ethyl acetate, 1.07 g/mL) and disopropylethylamine (130 μL, 0.75 mmol) were combined in dichloromethane (0.6 mL) and stirred at room temperature for 3.5 hours. The reaction mixture was partitioned between ethyl acetate (15 mL) and water (15 mL). The aqueous layer was extracted with additional ethyl acetate and the organic layers were combined, washed with brine, dried over magnesium sulfate, filtered and then concentrated under reduced pressure. The resulting material was then purified by normal phase column chromatography (ISCO RediSep Gold 12 column, 0-100% (3:1 ethyl acetate:ethanol) in hexane. The product fractions were concentrated to give JQ1-PEG3-azide as a colorless oil (68 mg, 45% yield). ¹H NMR (500 MHz, CDCl₃) δ: 7.44 (d, 2H, J 8.3 Hz), 7.36 (d, 2H, J 8.4 Hz), 6.90 (bs, 1H), 4.68 (t, 1H, J 7.0 Hz), 3.75-3.69 (m, 8H), 3.63 (m 2H), 3.55 (m, 2H), 3.45-3.37 (m, 2H), 2.69 (s, 3H), 2.43 (s, 3H), 1.70 (s, 3H). ¹³C NMR (125 MHz, CDCl₃) δ:170.6, 163.9, 155.7, 149.9, 136.8, 136.7, 132.2, 130.9, 130.8, 130.5, 129.9, 128.7, 70.7, 70.7, 70.7, 70.4, 70.0, 69.8, 54.4, 50.7, 39.4, 39.2, 14.4, 13.1, 11.8. m/z HRMS found [M]⁺=601.2125, [C₂₇H₃₃ClN₈O₄S]⁺ requires 601.2125.

JQ1-PEG3-azide (11 mg, 0.02 mmol) and Ir-alkyne [generation 1] (21 mg, 0.02 mmol) and DIPEA (16 μL, 0.1 mmol) were combined in acetonitrile (0.2 mL) to give a hazy suspension. To this suspension was added a freshly prepared suspension of copper sulfate (1.4 mg, 0.005 mmol) and sodium ascorbate (3.3 mg, 0.02 mmol) in water (0.3 mL) which instantly resulted in a yellow solution. This reaction mixture was stirred at room temperature for 5 hours at which point it was diluted with 1.5 mL DMSO and purified by preparative HPLC (50-100% MeCN/water, 0.05% TFA over 10 minutes, 20 mL/min, LUNA 5 micron C18(2) 100 angstrom, 250×21.2 mm). The product fraction was lyophilized. Preparative HPLC (same conditions) was repeated and the product fraction was lyophilized to give JQ-1-PEG3-Ir (6 mg, 20% yield) as a yellow solid. ¹H NMR (500 MHz, MeOH-d₄) δ: 9.07 (s, 1H), 8.92 (s, 1H), 8.70 (s, 2H), 8.14-8.08 (m, 2H), 8.06 (s, 1H), 7.86-7.80 (m, 2H), 7.66 (d, J=10.4 Hz, 2H), 7.50-7.43 (m, 2H), 7.40 (dd, J=8.7, 3.9 Hz, 2H), 6.92-6.79 (m, 2H), 5.94-5.85 (m, 2H), 4.69-4.61 (m, 1H), 4.57 (q, J=4.5 Hz, 2H), 4.53-4.43 (m, 2H), 3.89 (t, J=4.8 Hz, 2H), 3.68-3.56 (m, 10H), 3.50-3.39 (m, 3H), 3.28 (dd, J=14.9, 5.2 Hz, 1H), 3.24 (d, J=2.5 Hz, 3H), 2.69 (d, J=3.2 Hz, 3H), 2.46 (s, 3H), 1.69 (dd, J=17.2, 3.9 Hz, 15H). ¹³C NMR (125 MHz, MeOH-d₄) δ: 171.32, 168.40, 166.33, 164.93, 164.59, 164.17, 162.20, 161.83, 161.67, 159.62, 159.51, 159.32, 156.29, 156.16, 155.51, 155.25, 151.03, 150.77, 149.66, 146.48, 144.21, 142.69, 136.67, 136.51, 132.09, 130.71, 130.57, 130.03, 128.41, 126.38, 126.13, 124.42, 123.22, 123.05, 122.80, 122.61, 122.54, 120.37, 113.94, 99.64, 99.42, 99.21, 77.45, 76.60, 70.12, 70.10, 69.94, 69.17, 68.99, 56.80, 53.63, 49.97, 49.92, 39.15, 37.18, 26.78, 26.74, 26.42, 26.38, 25.81, 13.00, 11.53, 10.17. ¹⁹F NMR (471 MHz, MeOH-d₄) δ: −61.73, −77.07, −103.74, −107.98.

m/z calcd. for C₇₃H₆₆ClF₁₀IrN₁₂O₁₀S (1719.3958 found 1719.3947 (M+H) and 860.2029 (M+2H)/2. LC retention time: 1.23 minutes using Acquity Single pole LCMS equipped with two channels (20 and 25 V). The flow rate is 0.6 ml/min on a 2.1×50 mm BEH 1.7 μM particle size column with gradient 5 to 100% MeCN for 1.8 min, hold for 0.2 min

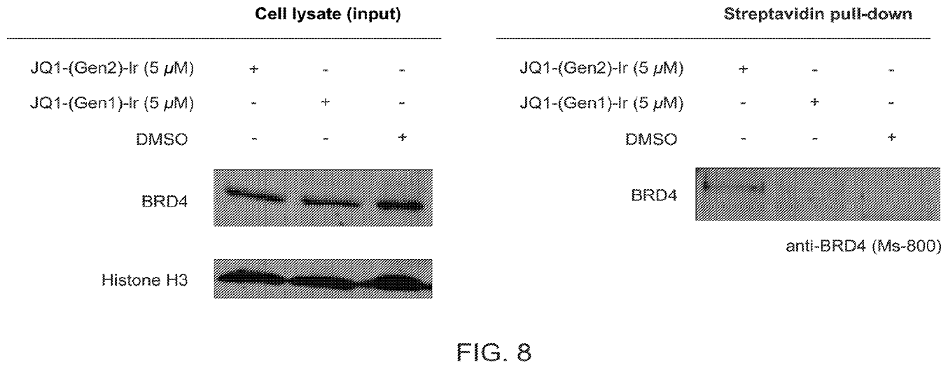

The cell permeable conjugate of Example 3 was provided for BRD4 labeling comparison in this example, and is referenced as JQ1-(Gen2)-Ir. Following the in-cell labelling protocol as described in Example 4. To HeLa cells in 12×10 cm plates at 80% confluency in DMEM with no phenol red (Gibco) (4 mL) was added JQ1-PEG3-Ir (Gen-2) (5 μM) (4 plates, A); JQ1-PEG3-Ir (Gen-1) (5 μM) (4 plates, B); and DMSO (4 plates, C). The plates were incubated at 37° C. for 3 hours and the media removed and replaced. Diazirine-PEG3-bioitin was added (250 μM) and the plates incubated at 37° C. for an additional 20 minutes. The plates were subsequently irradiated (without the lid) in the bioreactor at 450 nM for 20 minutes. Streptavidin enrichment and western blot performed as previously described. The results of the labeling are provided in FIG. 8. As show in the results, JQ1-(Gen1)-Ir lacked the ability to enter the cell and effectuate BRD4 labeling. In contrast, JQ1-(Gen2)-Ir entered the intracellular environment for BRD4 labeling.

### Example 8—Comparing Labelling Between (+)-JQ1 and (−)-JQ1 Conjugates

(−)-JQ1 has NO affinity for BRD-proteins and hence serves as a negative control.

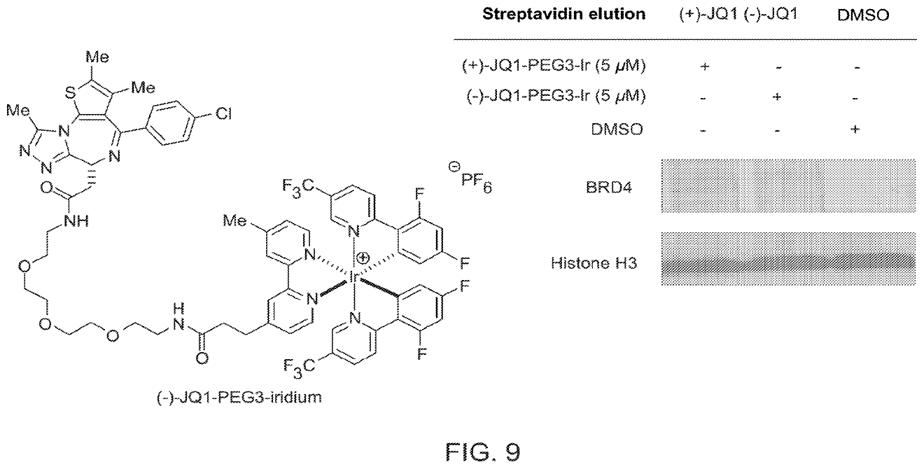

Following the in-cell labelling protocol as described above in Example 4. To HeLa cells in 12×10 cm plates at 80% confluency in DMEM with no phenol red (Gibco) (4 mL) was added (+)-JQ1-PEG3-Ir (Gen-2) (5 μM) (4 plates, A); (−)-JQ1-PEG3-Ir (Gen-2) (5 μM) (4 plates, B); and DMSO (4 plates, C). The plates were incubated at 37° C. for 3 hours and the media removed and replaced. Diazirine-PEG3-bioitin was added (250 μM) and the plates incubated at 37° C. for an additional 20 minutes. The plates were subsequently irradiated (without the lid) in the bioreactor at 450 nM for 20 minutes. Streptavidin enrichment and western blot performed as previously described. The results are provided in FIG. 9.

### Example 9—Selective Labeling of BRD4 Proteins with (+)-JQ1 Conjugate

### Proteomics Preparation and Isobaric Labelling:

The procedure carried out is identical to that of the in-cell labelling for western blot analysis of Example 4. To HeLa cells in 12×10 cm plates at 80% confluency in DMEM with no phenol red (Gibco) (4 mL) was added JQ1-PEG3-Ir (5 μM) (6 plates, A) and Ir-PEG3-NHBoc [referred to as Free-Ir during analysis] (5 μM) (6 plates, B). The plates were incubated at 37° C. for 3 hours and the media removed and replaced. Diazirine-PEG3-bioitin was added (250 μM) and the plates incubated at 37° C. for an additional 20 minutes. The plates were subsequently irradiated (without the lid) in the bioreactor at 450 nM for 15 minutes. The media was removed and the cells washed twice with cold DPBS (4° C.). The cells were resuspended in cold DPBS (4° C.), scraped and transferred into separate 15 mL falcon tube (2 plates per tube; 6 tubes in total). The cells were pelleted (1000 g for 5 minutes at 4° C.) and suspended in 2 mL of cold RIPA buffer containing PMSF (1 mM) and cOmplete EDTA free protease inhibitor (1×) (Roche). The lysed cells were incubated on ice for 5-10 minutes and sonicated (35%, 5×5 s with 30 s rest). The lysate was then centrifuged at 15×1000 g for 15 mins at 4° C. and the supernatant collected. The concentration of the cell lysate was measured by BCA assay and adjusted accordingly to a concentration of 1.5 mg/mL. Magnetic Streptavidin beads (NEB) were removed (350 μL per plex) and washed twice with RIPA (0.5 mL) (5 minutes incubation on a rotisserie). The beads were pelleted on a magnetic rack, diluted with the samples (1 mL) and incubated on a rotisserie at 4° C. overnight. The beads were pelleted on a magnetic rack, the supernatant removed, and a control sample from each plex (15 μL) and stored at −20° C. for later analysis. The beads were subsequently washed with 1×RIPA (0.5 mL), 3×1% SDS in DPBS (0.5 mL), 3×1M NaCl in DPBS (0.5 mL), 3×10% EtOH in DPBS and 1×RIPA (0.5 mL). The samples were incubated with each wash for 5 minutes prior to pelleting. The beads were resuspended in RIPA buffer (300 μL) and transferred to a new 1.5 mL Lo-bind tube.

The supernatant was removed and the beads washed with 3×DPBS (0.5 mL) and 3×NH₄HCO₃ (100 mM) (0.5 mL). The beads were re-suspended in 500 μL 6 M urea in DPBS and 25 μL of 200 mM DTT in 25 mM NH₄HCO₃ was added. The beads were incubated at 55° C. for 30 min. Subsequently, 30 μL 500 mM IAA in 25 mM NH₄HCO₃ was added and incubated for 30 min at room temperature in the dark. The supernatant was removed and the beads washed with 3×0.5 mL DPBS and 3×0.5 mL TEAB (50 mM). The beads were resuspended in 0.5 mL TEAB (50 mM) and transferred to a new protein LoBind tube, pelleted, and the supernatant removed. The beads were resuspended in 40 μL TEAB (50 mM) and 1.2 μL trypsin (1 mg/mL in 50 mM acetic acid) was added and the beads incubated overnight on a rotisserie at 37° C. After 16 hours, an additional 0.8 μL trypsin was added and the beads incubated for an additional 1 hour on a rotisserie at 37° C. Meanwhile, the TMT10 plex label reagents (0.8 mg) (Thermo) were equilibrated to room temperature and diluted with 41 μL of anhydrous acetonitrile (Optima grade; 5 min with vortexing) and centrifuged. The beads were subsequently pelleted and the supernatant transferred to the corresponding TMT-label.

| | | |
|---|---|---|
| A1: 127 N | B1: 128 C | C1: 130 N |
| A2: 127 C | B2: 129 N | C2: 130 C |
| A3: 128 N | B3: 129 C | C3: 131 |

The reaction was incubated for 2 hours at room temperature. The samples were quenched with 8 μL of 5% hydroxylamine and incubated for 15 minutes. All of the samples were pooled in a new Protein LoBind tune and quenched with TFA (16 μL, Optima). The samples were stored at −80° C. until proteomics were conducted. Samples were desalted and fractionated prior to running.

### LC-MS/MS/MS-Based Proteomic Analysis

Mass spectra were obtained using an Orbitrap Fusion at Princeton Proteomics Facility and analysed using MaxQuant. TMT labeled peptides were dried down in SpeedVac, re-dissolved in 300 μl of 0.1% TFA in water and fractionated into 8 fractions using Pierce™ High pH Reversed-Phase Peptide Fractionation Kit (#84868). Fractions 1, 4, and 7 were combined as sample 1. Fractions 2 and 6 were combined as sample 2. Fractions 3, 5, and 8 were combined as sample 3. Three combined samples were dried completely in a SpeedVac and resuspended in 20 μl 5% acetonitrile/water (0.1% formic acid (pH=3)). 2 μl (˜360 ng) was injected per run using an Easy-nLC 1200 UPLC system. Samples were loaded directly onto a 45 cm long 75 um inner diameter nano capillary column packed with 1.9 um C18-AQ resin (Dr. Maisch, Germany) mated to metal emitter in-line with an Orbitrap Fusion Lumos (Thermo Scientific, USA). Column temperature was set at 45° C. and two-hour gradient method with 300 nl per minute flow. The mass spectrometer was operated in data dependent mode with synchronous precursor selection (SPS)—MS3 method [Anal Chem. 2014, 86 (14), 7150-7158] with 120,000 resolution of MS1 scan (positive mode, profile data type, Intensity threshold 5.0e3 and mass range of 375-1600 m/z) in the Orbitrap followed by CID fragmentation in ion trap with 35% collision energy for MS2 and HCD fragmentation in Orbitrap (50,000 resolution) with 55% collision energy for MS3. MS3 scan range was set at 100-500 with injection time of 120 ms. Dynamic exclusion list was invoked to exclude previously sequenced peptides for 60 s and maximum cycle time of 2.5 s was used. Peptides were isolated for fragmentation using quadrupole (0.7 m/z isolation window). Ion-trap was operated in Rapid mode.

MS/MS/MS data was searched against 2018 Uniprot human protein database containing common contaminants (forward and reverse). Samples were set to three fractions and database search criteria were applied as follows: variable modifications set to methionine oxidation and N-terminal acetylation and deamidation (NQ), and fixed modifications set to cysteine carbamidomethylation, with a maximum of 5 modifications per peptide. Specific tryptic digestion (trypsin/P) with a maximum of 2 missed cleavages. Peptide samples were matched between runs. The maximum peptide mass was set to 6000 Da. The label minimum ration count was set to 2 and quantified using both unique and razor peptides. FTMS MS/MS match tolerance was set to 0.05 Da, and ITMS MS/MS match tolerance was set to 0.6 Da. All other settings were left as default.

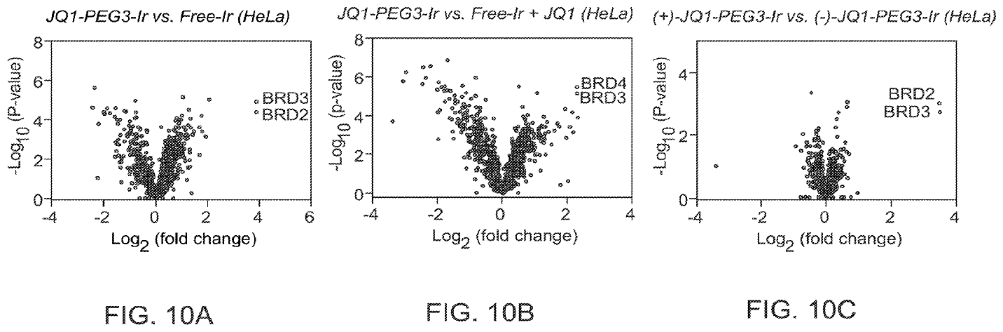

The proteinGroups.txt file was subsequently imported into Persues [Main: corrected reported intensities; the remaining entries left to default]. The rows were subsequently filtered by categorical column with ‘+’ values with matching rows removed via a reduced matrix based upon the following criteria, ‘only identified by site’, ‘reverse’, and ‘potential contaminant’. The resulting matrix was then transformed by log 2(x) and the column correlation verified to be >0.9. From the previous matrix, the rows were annotated (categorical annotation of rows) into their corresponding experiments (3×A, 3×B). The matrix was subsequently normalized (subtraction of columns), and the corresponding data plotted as a scatter graph (volcano plot). The FDR was determined by a 2-sample T-test (Benjamini-Hochberg). The results are provided in the volcano plots of FIGS. 10A-10C. As illustrated in FIGS. 10A-10C, the (+)-JQ1 conjugate resulted in significant enrichment of labelled proteins in the bromodomain family relative to the comparative conjugate species.

### Example 10—Cell Permeable Conjugate, Taxol-Ir

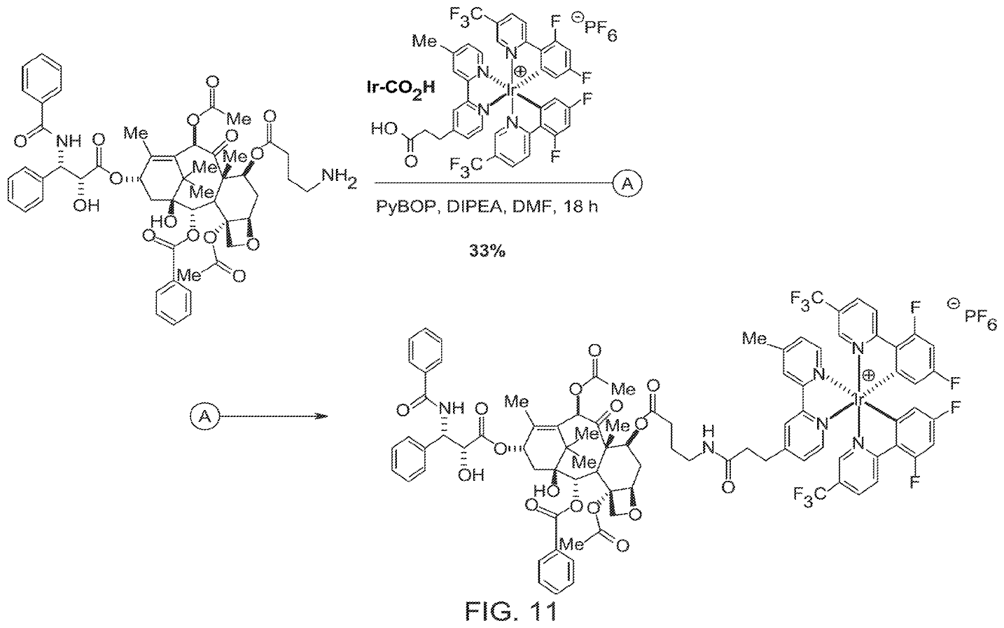

A cell permeable Taxol-Ir conjugate having structure described herein was produced according to the synthetic scheme of FIG. 11 and described below.

To a stirred solution of Ir-CO₂H (75 mg, 69 μmol), and PyBOP (55 mg, 105 μmol) in anhydrous DMF (1 mL) under N₂ in the dark was added DIPEA (30 μL, 172 μmol). The resulting mixture was stirred at room temperature for 10 minutes and a solution of Taxol-NH₂ (66 mg, 70 μmol) in anhydrous DMF (1 mL) was added dropwise. The reaction was stirred overnight, diluted with EtOAc, and quenched by the addition of saturated aqueous NaHCO₃. The aqueous phase was removed and the organic layer washed with additional saturated aqueous NaHCO₃, 5% aqueous citric acid, brine, and dried over Na₂SO₄. The solvent was removed in vacuo, and the crude material purified by silica column chromatography (gradient elution: 0 to 3% MeOH/CH₂Cl₂) and C8 reverse phase preparative HPLC (gradient elution: 30 to 100% MeCN/H₂O (0.1% formic acid)) to afford Taxol-iridium as a yellow solid (47 mg, 33%). ¹H NMR (500 MHz, CDCl₃) δ: 8.77 (d, J=7.3 Hz, 1H), 8.75 (s, 1H), 8.77-8.65 (m, 1H), 8.48 (t, J=10.5 Hz, 2H), 8.14-7.99 (m, 4H), 7.92-7.77 (m, 2H), 7.82 (d, J=7.3 Hz, 2H), 7.74 (t, J=7.3 Hz, 2H), 7.66-7.28 (m, 13H), 7.04-6.94 (m, 1H), 6.64 (t, J=9.4 Hz, 2H), 6.16 (s, 1H), 6.10 (t, J=8.4 Hz, 1H), 5.79-5.68 (m, 1H), 5.67-5.57 (m, 3H). 5.55-5.45 (m, 1H), 5.29 (s, 1H), 4.90 (d, J=9.6 Hz, 1H), 4.84 (d, J=3.6 Hz, 1H), 4.27 (d, J=8.9 Hz, 1H), 4.15 (d, J=7.9 Hz, 1H), 3.87 (d, J=7.9 Hz, 1H), 3.16 (app. s, 4H), 2.95-2.58 (m, 7H), 2.58-2.50 (m, 1H), 2.35 (app. s, 3H), 2.26-2.09 (m, 5H), 1.86-1.63 (m, 7H), 1.25 (app. s, 3H), 1.16 (s, 3H), 1.13 (s, 3H). ¹³C NMR (125 MHz, CDCl₃) δ: 202.03, 172.7, 172.5, 171.5 (d, J=3.2 Hz), 170.5, 169.6, 169.5, 168.2-168.0 (m), 167.3, 167.0, 165.0 (dd, J=262.5, 13.0 Hz), 262.7 (dd, J=263.7, 13.0 Hz), 157.7, 153.4-155.2 (m), 155.1-155.0 (m), 154.8-154.6 (m), 149.7, 149.3, 145.1-144.8 (m), 140.8 (d, J=2.6 Hz), 138.7 (d, J=2.0 Hz), 136.8-136.6 (m), 134.1 (d, J=2.1 Hz), 133.9, 132.8, 131.8, 130.3, 130.1 (d, J=6.1 Hz), 129.8, 129.3, 128.9, 128.8, 128.7, 128.1, 127.4, 126.4, 126.2, 123.9 (t, J=21.3 Hz), 122.7 (d, J=9.1 Hz), 120.6 (d, J=9.1 Hz), 114.2 (dd, J=16.5, 6.7 Hz), 100.1 (td, J=27.0, 9.8 Hz), 84.1, 81.0, 78.6, 76.5, 75.4, 74.5, 73.5, 71.6, 71.5, 71.5, 56.2, 55.9, 55.8, 53.6, 47.1, 43.3, 38.8, 35.5, 35.4, 35.3, 33.4, 31.2, 29.8, 26.5, 26.4, 23.8, 23.8, 22.7, 21.6, 21.0, 20.9, 14.6, 11.0. ¹⁹F NMR (376 MHz, CDCl₃) δ: −62.7 (d, J=5.6 Hz), −62.8 (d, J=5.0 Hz), −71.0, −72.9, −101.3-−101.5 (m), −105.7-−105.9 (m). m/z HRMS found [M]⁺=1871.51783 (100), 1872.51899 (89), 1869.51134 (55), 1870.51373 (55), 1873.51932 (52), 1874.52130 (22), [C₈₉H₈₀F₁₀IrN₆O₁₆]⁺ requires 1871.50949 (100), 1872.51284 (96), 1869.50715 (60), 1870.51051 (57), 1873.51620 (46), 1874.51955 (14). HPLC (Vydac 218TP C18 HPLC, gradient: 0-90% MeCN/H₂O (0.1% TFA) 10 minutes, 5 minutes 90% MeCN (0.1% TFA), 1 mL/min, 254 nm): tᵣ=13.3 min.

### Example 11—Intracellular Microenvironmental Mapping

### In-Cell Labelling:

To MCF-7 cells in 10 clear 10 cm plate at 80% confluency in RPMI 1640 with no phenol red (Gibco) (4 mL) was added Taxol-Ir (Example 10) (20 μM) (5 plates, A) and Ir-dF(CF₃)(dMebpy)PF₆ [referred to as Free-Ir during analysis] (2 μM) (5 plates, B). The plates were incubated at 37° C. for 3 hours and the media removed and replaced. N-(4-(3-(trifluoromethyl)-3H-diazirin-3-yl)benzyl)hex-5-ynamide was added (250 μM) and the plates incubated at 37° C. for an additional 20 minutes. The plates were subsequently irradiated (without the lid) in the bioreactor at 450 nM for 20 minutes. The plate was subsequently irradiated (without the lid) in the Merck bioreactor at 450 nM for 15 minutes. The media was subsequently removed and the cells gently washed with cold DPBS (2×5 mL), the cells scraped (in 5 mL cold DPBS), combined, and pelleted (1000 g for 5 minutes at 4° C.). The supernatant was removed and the cells suspended in 1 mL of cold lysis buffer (1% SDS in 10 mM HEPES, 150 mM NaCl, 1.3 mM MgCl₂) containing PMSF (1 mM) and cOmplete EDTA free protease inhibitor (Roche). The lysed cells were incubated on ice and sonicated (35%, 4×5 s with 30 s rest). The lysate was then centrifuged at 15×1000 g for 15 mins at 4° C. and the supernatant collected. The concentration of the cell lysate was measured by BCA assay (typically 3 mg/mL).

### CuAAC Reaction:

Click-cocktail for 3 plexes: In a 0.5 mL Lo-bind tube, 6.2 μL 500 mM CuSO₄ was added to 62 μL 100 mM THPTA and vortexed. Subsequently, 15.5 μL 5 mM biotin-PEG7-azide (broadpharm) was added, followed by 15.5 μL of freshly prepared 1M sodium ascorbate (Important: addition of reagents in that order).

To the cell-lysate (1 mL) in a 1.5 mL Lo-bind tube was added 32 μL of the click-cocktail. The resulting solution was vortexed and incubated on a rotisserie at room temperature for 1 hour and quenched by the addition of 5 μL 250 mM Na₄EDTA. The mixture was cooled to 0° C., transferred to a 15 mL tube, and diluted with 4.2 mL ice-cold acetone. The samples were precipitated at −20° C. overnight (3 hours was also found to be satisfactory), centrifuged at 4.5×1000 g for 20 mins at 4° C., and the supernatant removed. The pellet was fully resuspended in ice-cold methanol (1 mL) by sonication (2 s at 20%) and incubated at −20° C. for 30 minutes. After such time, the mixture was centrifuged at 4.5×1000 g for 20 mins at 4° C. and the supernatant removed. The procedure was repeated. The pellet was allowed to air dry for 20 mins at room temperature and redissolved in 300 μL 1% SDS (1 h at room temperature) and heated for 5 mins at 95° C. The samples were cooled and diluted with 900 μL RIPA buffer. 250 μL of streptavidin magnetic beads (Thermo Fisher, cat. 88817) were added to Protein LoBind microcentrifuge tubes (Eppendorf, cat. 022431081) and washed 2× with 1 mL RIPA Buffer (Thermo Fisher, cat. 89900). Approximately 1.0 mg of cell lysate was added to the pre-washed streptavidin magnetic beads and incubated for 3 hours at room temperature. A magnetic rack was used to pellet the beads and remove the lysate supernatant. The beads were sequentially washed 3× with each of the following: 1 mL of 1% SDS, 1 mL of 1M NaCl, and 1 mL of 10% EtOH, all prepared in 1×DPBS and incubating for 5 min in between washes. A final wash was done with 1 mL RIPA Buffer. The beads were then resuspended in 30 μL of 4× Laemmli sample buffer (Boston BioProducts, cat. BP-110R) containing 20 mM DTT and 25 mM biotin. Beads were heated for 10 min at 95° C. and were then placed on the magnetic rack. The supernatant was transferred to a new Protein LoBind microcentrifuge tube and stored at −80° C. Quantitative proteomic sample preparation and analysis was performed by IQ Proteomics (Cambridge, Mass.).

For LC-MS analysis at IQ Proteomics, mass spectra were acquired on an Orbitrap Fusion Lumos coupled to an EASY nanoLC-1000 (or nanoLC-1200) (Thermo Fisher) liquid chromatography system. Approximately 2 μg of peptides were loaded on a 75 m capillary column packed in-house with Sepax GP-C18 resin (1.8 m, 150 Å, Sepax) to a final length of 35 cm. Peptides were separated using a 110-minute linear gradient from 8% to 28% acetonitrile in 0.1% formic acid. The mass spectrometer was operated in a data dependent mode. The scan sequence began with FTMS1 spectra (resolution=120,000; mass range of 350-1400 m/z; max injection time of 50 ms; AGC target of 1·106; dynamic exclusion for 60 seconds with a +/−10 ppm window). The ten most intense precursor ions were selected for MS2 analysis via collisional-induced dissociation (CID) in the ion trap (normalized collision energy (NCE)=35; max injection time=100 ms; isolation window of 0.7 Da; AGC target of 1.5.104). Following MS2 acquisition, a synchronous-precursor-selection (SPS) MS3 method was enabled to select eight MS2 product ions for high energy collisional-induced dissociation (HCD) with analysis in the Orbitrap (NCE=55; resolution=50,000; max injection time=86 ms; AGC target of 1.4·10⁵; isolation window at 1.2 Da for +2 m/z, 1.0 Da for +3 m/z or 0.8 Da for +4 to +6 m/z). All mass spectra were converted to mzXML using a modified version of ReAdW.exe. MS/MS spectra were searched against a concatenated 2018 human Uniprot protein database containing common contaminants (forward+reverse sequences) using the SEQUEST algorithm. Database search criteria are as follows: fully tryptic with two missed cleavages; a precursor mass tolerance of 50 ppm and a fragment ion tolerance of 1 Da; oxidation of methionine (15.9949 Da) was set as differential modifications. Static modifications were carboxyamidomethylation of cysteines (57.0214) and TMT on lysines and N-termini of peptides (229.1629). Peptide-spectrum matches were filtered using linear discriminant analysis and adjusted to a 1% peptide false discovery rate (FDR).

All bioinformatic analysis of LC-MS/MS data was performed in the R statistical computing environment. Peptide level abundance data was used to identify the number of peptides corresponding to a protein in the experiment. Any protein with a single peptide quantification was removed to reduce the possibility that outliers would affect downstream proximal calls. Peptide level abundance data was then normalized to the summed total abundance for each sample separately. These totals were then averaged, and each normalized protein abundance value was multiplied by this average to rescale abundance data. Peptide level data was then merged to protein level data by taking the median of all peptides corresponding to a protein. Proteins were then filtered to remove any known contaminants identified from the database search and proteins which are known antibody contaminants (e.g. having IGK, IGK, or IGH present in the gene symbol and Immunoglobulin present in the Uniprot description). Data were then filtered to remove PRNP, a protein which is a known false positive consistently detected across almost all experiments. Protein abundances were log 2 transformed and subjected to linear modeling analysis with Limma. Limma utilizes an empirical Bayes approach that allows for a realistic distribution of biological variance with small sample sizes per group. This program further utilizes the full dataset to shrink the observed sample variances towards a pooled estimate. This borrowing of variance information across proteins allows for a more accurate estimate of true variance, and improved power to detect real differences between groups. For each protein, abundance data was fit to a linear model with the experimental group as the input variable using the lmFit function. The log₂FC values were estimated and p-values calculated for significance. P-values were then corrected for multiple comparisons using the false discovery rate (FDR) method by Benjamini and Hochberg. Volcano plots were generated in R with the ggplot2 library. Log₂FC and p-value estimates from Limma were subset to those reaching a specified log 2FC cutoff. Proteins were colored based on whether they fell above or below the log 2-fold cutoff threshold and were statistically significant (FDR corrected p-value of <0.05).

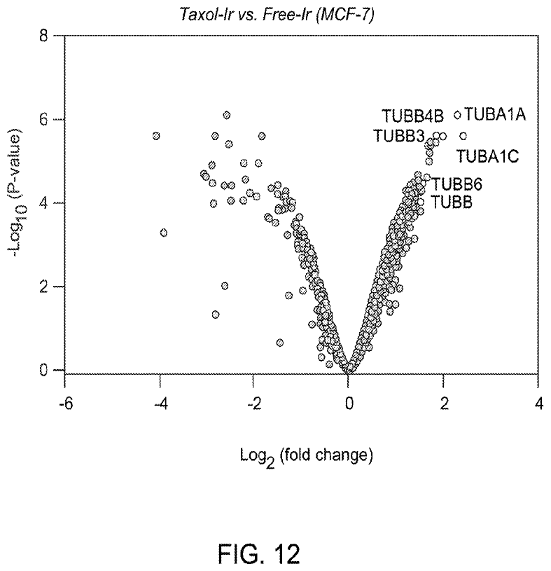

FIG. 12 provides a volcano plot of significance vs. fold enrichment for targeted tubulin proteins in MCF-7 cells using the cell permeable conjugate of Example 10 for labeling

### Example 12—Confocal Microscopy

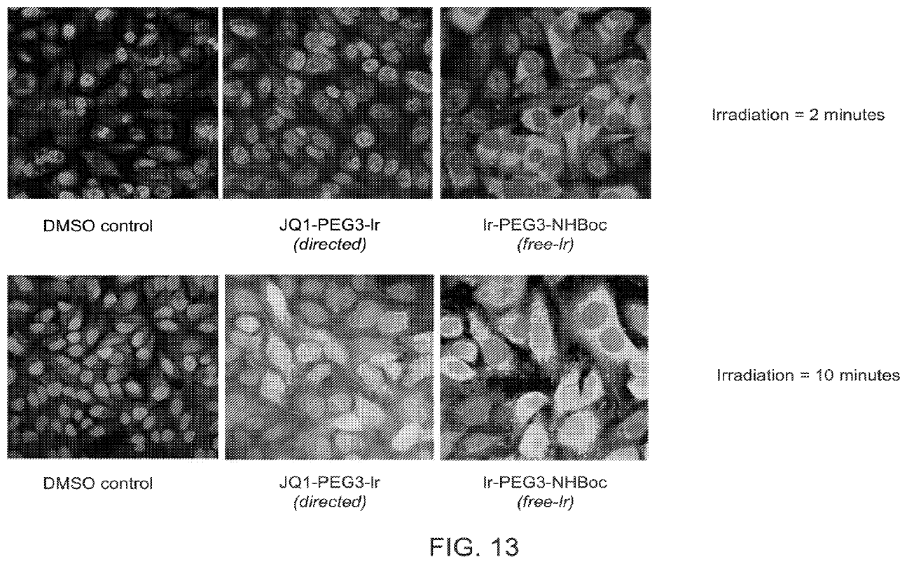

HeLa cells were plated onto 35 mm glass-bottom microscopy dishes with DMEM (no phenol red) and treated with (+)-JQ1-PEG3-Ir (Example 3) (5 μM), Ir-PEG3-NHBoc [referred to as Free-Ir] (5 μM), and DMSO. The plates were incubated at 37° C. for 3 hours and the media removed and replaced. Diazirine-PEG3-bioitin was added (250 μM) and the plates incubated at 37° C. for an additional 20 minutes. The plates were subsequently irradiated (without the lid) in the bioreactor at 450 nM for different time periods. The media was removed, and the cells were washed with PBS. Cells were then fixed with 400 μL of 4% paraformaldehyde in PBS for 20 min at 37 C. Cells were washed 3× with PBS and permeabilized with 400 μL of 0.1% triton X-100 in PBS for 20 min at RT. Cells were washed with PBS and blocked with 400 μL of 2% BSA in PBS for 20 min at RT. Cells were washed 3× with PBS and incubated with 400 μL of Streptavidin-Alexa Fluor 488 diluted 1:500 and Hoechst diluted 1:10,000 in PBS. Confocal microscopy was performed at 40× magnification using a Nikon A1/HD25 microscope (Nikon Instruments, Inc., Melville, N.Y.). The image of FIG. 13 are representative of the multiple cross-sectional images taken during each session.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A transition metal complex of Formula I: 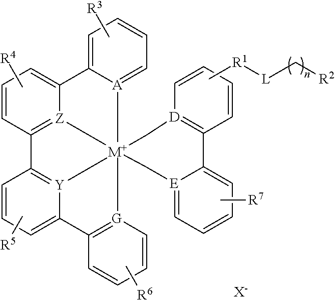
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is a linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

2. The transition metal complex of claim 1, wherein M is a platinum group metal.

3. The transition metal complex of claim 2, wherein M is iridiuim.

4. The transition metal complex of claim 1 having an absorption spectrum in the visible region of the electromagnetic spectrum.

5. The transition metal complex of claim 1, wherein R² is selected to comprise a moiety for coupling a biomolecule.

6. The transition metal complex of claim 5, wherein the R² is a click chemistry moiety.

7. The transition metal complex of claim 6, wherein the click chemistry moiety is selected from the group consisting of BCN, DBCO, TCO, tetrazine, alkyne, and azide.

8. The transition metal complex of claim 7, wherein the biomolecule is an antibody.

9. The transition metal complex of claim 1, wherein the transition metal complex is cell permeable.

10. The transition metal complex of claim 1, having aqueous solubility of 1 μM to 150 μm at 0.2% DMSO in pure water.

11. A conjugate comprising:
a transition metal complex coupled to a biomolecular binding agent, wherein prior to coupling to the biomolecular binding agent, the transition metal complex is of Formula I: 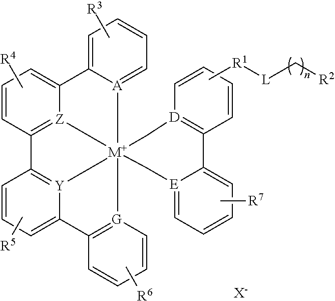
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is a linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

12. The conjugate of claim 11, wherein the transition metal complex and biomolecular binding agent are coupled via click chemistry.

13. The conjugate of claim 11, wherein M is a platinum group metal.

14. The conjugate of claim 11, wherein the transition metal complex has an absorption spectrum in the visible region of the electromagnetic spectrum.

15. The conjugate of claim 11, wherein the conjugate is cell permeable.

16. The conjugate of claim 11, having aqueous solubility of 1 μM to 150 μm at 0.2% DMSO in pure water.

17. A system for proximity labeling comprising:
a protein labeling agent; and
a transition metal catalyst, wherein the transition metal catalyst has electronic structure permitting electron transfer to the protein labeling agent to provide a reactive intermediate, and wherein the transition metal catalyst is of Formula I: 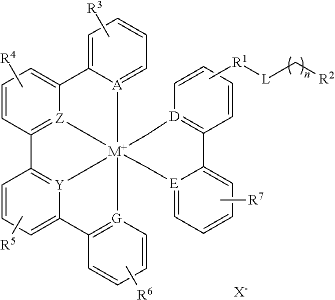
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is a linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

18. The system of claim 17, wherein the electron transfer originates from an excited state of the catalyst electronic structure.

19. The system of claim 18, wherein the electron transfer originates from a triplet state of the catalyst electronic structure.

20. The system of claim 17, wherein the reactive intermediate has a diffusion radius of 1-500 nm.

21. The system of claim 20, wherein the diffusion radius is 1-10 nm.

22. The system of claim 17, wherein the transition metal catalyst is coupled to a biomolecular binding agent.

23. The system of claim 22, wherein the biomolecular binding agent comprises a peptide, protein, sugar, small molecule or nucleic acid.

24. The system of claim 22, wherein the transition metal complex and biomolecular binding agent are coupled via click chemistry.

25. The system of claim 17, wherein the protein labeling agent is a diazirine.

26. The system of claim 25, wherein the diazirine comprises a molecular marker.

27. The system of claim 25, wherein the reactive intermediate is a carbene.

28. The system of claim 22, wherein the transition metal catalyst is cell permeable.

29. A method of proximity labeling comprising:
providing a protein labeling agent and conjugate comprising a transition metal catalyst coupled to a biomolecular binding agent;
activating the protein labeling agent to a reactive intermediate with the transition metal catalyst; and
coupling the reactive intermediate to a protein in a cellular environment, wherein the transition metal complex is of Formula I: 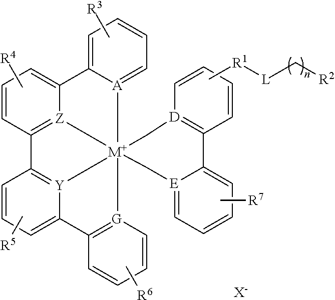
wherein M is a transition metal;
wherein A, D, E, G, Y and Z are independently selected from C and N;
wherein R³-R⁷ each represent one to four optional ring substituents, each of the one to four optional ring substituents independently selected from the group consisting of alkyl, heteroalkyl, haloalkyl, haloalkenyl, 3halo, hydroxy, alkoxy, amine, amide, ether, —C(O)O⁻, —C(O)OR⁸, and —R⁹OH, wherein R⁸ is selected from the group consisting of hydrogen and alkyl, and R⁹ is alkyl;
wherein R¹ is selected from the group consisting of a direct bond, alkylene, alkenylene, cycloaklylene, cycloalkenylene, arylene, heteroalkylene, heteroalkenylene, heterocyclene, and heteroarylene;
wherein L is a linking moiety selected from the group consisting of amide, ester, sulfonamide, sulfonate, carbamate, and urea; and
R² is selected from the group consisting of alkyne, amine, protected amine, azide, hydrazide, aryl, heteroaryl, cycloalkyl, cycloalkenyl, cycloalkylnyl, heterocyclyl, hydroxy, carboxyl, halo, alkoxy, maleimide, —C(O)H, —C(O)OR⁸, —OS(O₂)R⁹, thiol, biotin, oxyamine, and haloalkyl, wherein R⁸ and R⁹ are independently selected from the group consisting of alkyl, haloalkyl, aryl, haloaryl, N-succinimidyl, and N-succinimidyl ester; and wherein X⁻ is a counterion, and n is an integer from 0 to 20.

30-45. (canceled)
